(19) United States
(12) Patent Application Publication
(10) Pub. No.: US 2026/0294516 A1
(43) Pub. Date: Oct. 1, 2026

(54) TECHNOLOGIES FOR MONITORING OPERATION OF A SURGICAL INSTRUMENT

(71) Applicant: Cilag GmbH International, Zug (CH)

(72) Inventor: Jose Luis De Cordoba Matilla, Cincinnati, OH (US)

(21) Appl. No.: 19/381,545

(22) Filed: Nov. 6, 2025

Related U.S. Application Data
(60) Provisional application No. 63/780,379, filed on Mar. 30, 2025.

(51) Int. Cl. A61B 18/14; A61B 18/00; A61B 34/20; A61B 90/00

(52) U.S. Cl. CPC A61B 18/1445; A61B 34/20; A61B 2018/00208; A61B 2018/00642; A61B 2034/2051; A61B 2034/2055; A61B 2034/2065; A61B 2090/3937

(57) ABSTRACT

A surgical instrument for performing an energy-based surgical procedure includes an end effector, a trigger assembly, and a position sensing circuit. The end effector includes a jaw assembly movable between an open state and a closed state. The trigger assembly is operable to move the jaw assembly of the end effector between the open state and the closed state. The position sensing circuit includes a position sensor and a position indicator. The position sensor is configured to sense movement of the position indicator and generate sensor data indicative of a position of the jaw assembly based on the sensed movement of the position indicator. A method for monitoring operation of the surgical instrument is also disclosed.

This application claims the benefit of and priority to U.S. Patent Application No. 63/780,379, entitled “TECHNOLOGIES FOR ROTATING ELECTRICAL CONTACTS ON SURGICAL DEVICES TO DELIVER ENERGY TO TISSUE,” which was filed on Mar. 30, 2025, and which is incorporated herein by reference in its entirety.

### TECHNICAL FIELD

The present disclosure relates generally to energy-based surgical instruments and, more particularly, to harmonic and/or electrosurgical surgical instruments.

### BACKGROUND

Energy-based surgical instruments are finding increasingly widespread applications in surgical procedures by virtue of their unique performance characteristics. Depending upon specific device configurations and operational parameters, energy-based surgical instruments can provide both transection of tissue and hemostasis of the tissue by coagulation, which may reduce or otherwise minimize patient trauma. Depending on the particular application, energy-based surgical instruments may utilize different surgical technologies including, for example, ultrasonic and/or electro-surgical (e.g., radio frequency (RF)) technologies.

A typical ultrasonic surgical instrument may include a handpiece containing an ultrasonic transducer and an elongated shaft assembly having a distally mounted end effector to effect the cutting and sealing of tissue. For example, the end effector may include a jaw assembly having an ultrasonic blade and a clamp arm, which may include a non-stick tissue pad or similar bed to receive the ultrasonic blade. In some cases, the elongated shaft assembly may be permanently affixed to the handpiece. In other cases, the elongated shaft assembly may be detachable from the handpiece, as in the case of a disposable shaft assembly or a shaft assembly that is interchangeable between different handpieces. In use, the end effector transmits ultrasonic energy to tissue brought into contact with the ultrasonic blade of the end effector to realize the cutting and sealing action. Such ultrasonic surgical devices may be configured for open surgical use, laparoscopic, and/or endoscopic surgical procedures including robotic-assisted procedures.

Ultrasonic energy cuts and coagulates tissue using temperatures lower than those used in electro-surgical procedures. Vibrating at high frequencies (e.g., 55,500 times per second), the ultrasonic blade denatures protein in the tissue to form a sticky coagulum. Pressure exerted on tissue by the ultrasonic blade surface collapses blood vessels and allows the coagulum to form a hemostatic seal. A surgeon can control the cutting speed and coagulation by the force applied to the tissue by the end effector, the time over which the force is applied, and the selected excursion level of the end effector.

In electro-surgical instruments, one or more electrodes are incorporated into the end effector and configured to apply therapeutic electrical current to the patient's tissue to create a hemostatic seal. In electro-surgical instruments that do not include a harmonic mode (i.e., do not include a harmonic blade), the end effector may be embodied as two clamp arms or jaws. In such embodiments, the electro-surgical instrument may include a separate mechanical knife or blade for cutting the tissue after the creation of the hemostatic seal, which may be incorporated into the elongated shaft attached to the end effector. In bi-polar embodiments, an active electrode may be attached to one of the clamp arms of the end effector and configured to introduce an electrical current into the tissue, which is received by a return electrode attached to the other clamp arm of the end effector (or as the blade itself in embodiments including a harmonic mode). Conversely, in mono-polar embodiments, the return electrode (e.g., a “grounding pad”) may be separate from the electro-surgical instrument and located on a different part of the body of the patient. In some embodiments, the electro-surgical instrument may also be configured to apply a sub-therapeutic electrical current to the patient's tissue, which may be used for sensing purposes (e.g., measuring tissue impedance).

Electro-surgery forms hemostatic seals by generating heat in the tissue via the introduced electrical energy, which is embodied as radio frequency (“RF”) energy. The particular frequency employed can vary based on the intended use of the electro-surgical instrument within the range of about 100 kHz to 1 MHz, although higher frequencies can be employed in some embodiments. Additionally, sub-therapeutic frequencies may be used in some situations for purposes other than hemostatic sealing, such as performing various electrical measurements on the tissue.

Some surgical instruments may include one or more rotating or otherwise movable parts. In such surgical instruments, an electrical connection may be required to be maintained across or through such rotating movable part. However, a rotating or movable electrical connections can be difficult to design to achieve a robust connection and reliable assembly. For example, under certain circumstances, typical rotating or movable electrical connections or contacts may be prone to failure, which can result in open circuits and failure of energy delivery to the tissue.

It should be appreciated that some energy-based surgical instruments may employ dual or multi-modal technologies for the transection and/or hemostasis of patient tissue. For example, in some cases, an energy-based surgical instrument may include both ultrasonic and electro-surgical capabilities (e.g., by utilizing the ultrasonic blade as an electrode for the electro-surgery mode), which increases the surgical options provided by the surgical instrument to the surgeon.

### SUMMARY

According to an aspect of the present disclosure, a surgical instrument for performing an energy-based surgical procedure may include an end effector, a trigger assembly, and a position sensing circuit. The end effector may have a jaw assembly movable between an open state and a closed state. The trigger assembly may be operable to move the jaw assembly of the end effector between the open state and the closed state. The position sensing circuit may include a position sensor and a position indicator. The position sensor is configured to sense movement of the position indicator and generate sensor data indicative of a position of the jaw assembly based on the sensed movement of the position indicator.

In some embodiments, the surgical instrument may further include a controller configured to determine the position of the jaw assembly based on the sensor data and control a function of the surgical instrument based on the determined position of the jaw assembly. Additionally or alternatively, the surgical instrument may further include a communication circuit configured to transmit the sensor data to a generator remote from the surgical instrument.

Additionally, in some embodiments, the position sensor of the position sensing circuit may be embodied as a camera configured to monitor movement of the position indicator. In such embodiments, the position indicator may be embodied as indica located in view of the camera. The indicia may include a marking located on an inner shaft of the surgical instrument and at least a portion of the marking may be covered from the view of the camera when the trigger assembly is operated to move the jaw assembly of the end effector.

In some embodiments, the trigger assembly may further include a yoke configured to move in response to operation of the trigger assembly to move the jaw assembly of the end effector. In such embodiments, the position indicator may be embodied as a yoke arm extending upwardly from a yoke body of the yoke. The yoke arm may be in view of the camera and may move within the view of the camera in response to operation of the trigger assembly. The position of the yoke arm within the view of the camera may be indicative of an amount of clamp force of the jaw assembly of the end effector.

Additionally, in some embodiments, the surgical instrument may further include an inner shaft and the trigger assembly may further include a yoke configured to move in response to operation of the trigger assembly to move the jaw assembly of the end effector. In such embodiments, the position indicator may be embodied as a first position indicator, and the first position indicator may include a marking located on the inner shaft. At least a portion of the marking may be covered from the view of the camera when the trigger assembly is operated to move the jaw assembly of the end effector. The surgical instrument may further include a second position indicator. The second position indicator may be embodied as a yoke arm extending upwardly from a yoke body of the yoke. The yoke arm may be in view of the camera and may move within the view of the camera in response to operation of the trigger assembly. The position of the yoke arm within the view of the camera may be indicative of an amount of clamp force of the jaw assembly of the end effector.

In some embodiments, the position sensor of the position sensing circuit may be embodied as an optocoupler configured to monitor movement of a reflective surface. For example, in some embodiments, the position sensor may include a sensor body, a first optocoupler mounted on a first side of the sensor body and a second optocoupler mounted on a second side of the sensor body. In such embodiments, the position indicator may include a first reflective ring mounted to an internal sleeve and in view of the first optocoupler and a second reflective ring mounted to an internal shaft and in view of the second optocoupler. The first reflective ring may be configured to move relative to the first optocoupler when the trigger assembly is operated to move the jaw assembly of the end effector, and the first optocoupler may be configured to sense a distance to the first reflective ring. The second reflective ring may be configured to move relative to the second optocoupler when the trigger assembly is operated to move the jaw assembly of the end effector, and the second optocoupler may be configured to sense a distance to the second reflective ring.

Additionally, in such embodiments, the trigger assembly may further include a yoke configured to move in response to operation of the trigger assembly to move the jaw assembly of the end effector. The position sensor may further include a third optocoupler mounted to the first side of the sensor body, and the position indicator may be embodied as a reflective surface of the yoke. The third optocoupler may be configured to sense a distance to the reflective surface of the yoke.

In some embodiments, the position sensor of the position sensing circuit may be embodied as a magnetic sensor and the position indicator may be embodied as a magnet. In such embodiments, the jaw assembly may include a jaw clamp and a harmonic blade. One of the magnetic sensor and the magnet may be attached to the jaw clamp and the other one of the magnetic sensor and the magnet may be attached to the harmonic blade.

According to another aspect of the present disclosure, a method may include sensing, by a position sensor, movement of a position indicator during operation of a trigger assembly of a surgical instrument to move the jaw assembly between an open state and a closed state; generating, by the position sensor, sensor data indicative of a position of the jaw assembly based on the sensed movement of the position indicator; and determining, by a controller of the surgical instrument, the position of the jaw assembly based on the sensor data. The position sensor and the position indicator may be attached to the surgical instrument.

In some embodiments, the position sensor of the position sensing circuit may be embodied as a camera configured to monitor movement of the position indicator, and the position indicator may be embodied as indica located in view of the camera. In such embodiments, the indicia may include a marking located on an inner shaft of the surgical instrument. At least a portion of the marking may be covered from the view of the camera when the trigger assembly is operated to move the jaw assembly of the end effector.

Additionally, in some embodiments, the position sensor of the position sensing circuit may be embodied as an optocoupler configured to monitor movement of a reflective surface. For example, the position sensor may be embodied as a sensor body, a first optocoupler mounted on a first side of the sensor body and a second optocoupler mounted on a second side of the sensor body.

In such embodiments, the position indicator may include a first reflective ring mounted to an internal sleeve and in view of the first optocoupler and a second reflective ring mounted to an internal shaft and in view of the second optocoupler. The first reflective ring may be configured to move relative to the first optocoupler when the trigger assembly is operated to move the jaw assembly of the end effector. The first optocoupler may be configured to sense a distance to the first reflective ring. The second reflective ring may be configured to move relative to the second optocoupler when the trigger assembly is operated to move the jaw assembly of the end effector. The second optocoupler may be configured to sense a distance to the second reflective ring.

Additionally, in some embodiments, the jaw assembly may include a jaw clamp and a harmonic blade. The position sensor of the position sensing circuit may include a magnetic sensor attached to one of the jaw clamp and the harmonic blade. The position indicator may include a magnet attached to the other one of the jaw clamp and the harmonic blade.

### BRIEF DESCRIPTION OF THE DRAWINGS

The detailed description particularly refers to the following figures, in which:

FIG. 1 is a simplified diagram of an embodiment of a system for performing an energy-based surgical procedure;

FIG. 2 is a perspective view of an embodiment of an energy-based surgical instrument of the system of FIG. 1;

FIG. 3 is a side elevation view of a jaw assembly of an end effector of the surgical instrument of FIG. 2 including an ultrasonic blade and in an open state;

FIG. 4 is a side elevation view of the jaw assembly of the end effector of the surgical instrument of FIG. 2 including an ultrasonic blade and in a closed state;

FIG. 5A is a perspective view of another embodiment of the end effector of the surgical instrument of FIG. 2 including an electrode on a lower jaw clamp of the jaw assembly;

FIG. 5B is a perspective view of another embodiment of the end effector of the surgical instrument of FIG. 2 including two jaw clamps, each having an electrode attached thereto;

FIG. 6 is an exploded view of the surgical instrument of FIG. 2;

FIG. 7 is a block diagram of a control circuit of the surgical instrument of FIG. 2;

FIG. 8 is a block diagram of another embodiment of a control circuit of the surgical instrument of FIG. 2 including a position sensing circuit;

FIG. 9 is a perspective view of the surgical instrument of FIG. 8 showing internal components of the surgical instrument and including an embodiment of the position sensing circuit having a camera and a marking attached to internal components of the surgical instrument;

FIG. 10 is a side view of the internal components of the surgical instrument of FIG. 9 showing the marking defined on an inner shaft of the surgical instrument and in view of the camera;

FIG. 11 is another side view of the internal components of the surgical instrument of FIG. 10 during operation of a trigger assembly of the surgical instrument causing the marking defined on the inner shaft to be partially covered by an internal sleeve of the surgical instrument;

FIG. 12 is a perspective view of another embodiment of a position sensor of the position sensing circuit of FIG. 8 including a pair of optocouplers;

FIG. 13 is side elevation view of the surgical instrument of FIG. 8 showing internal components of the surgical instrument and including the position sensor of FIG. 12;

FIG. 14 is perspective view of the surgical instrument of FIG. 13 including the position sensor of FIG. 12 and a position sensing circuit of FIG. 8 embodied as a reflective ring attached to an internal sleeve of the surgical instrument;

FIG. 15 is another perspective view of the surgical instrument of FIG. 8 including the position sensor of FIG. 12 having a third optocoupler configured to sense a reflective surface of a yoke of the surgical instrument;

FIG. 16 is a perspective view of the yoke and the internal sleeve of the surgical instrument of FIG. 15 including the reflective ring of FIG. 14 attached to the internal sleeve and the reflective surface of the yoke;

FIG. 17 is a perspective view of an internal shaft of the surgical instrument of FIG. 15 showing another reflective ring attached to the internal shaft;

FIG. 18 is a side perspective view of a jaw assembly of the surgical instrument of FIG. 8 including a position sensing circuit having a magnet attached to a jaw clamp of the jaw assembly and a magnetic sensor attached to a blade of the jaw assembly;

FIG. 19 is a proximal cross-section view of a shaft of the surgical instrument of FIG. 8 showing internal wiring for electrical connection to the position sensing circuit of the surgical instrument of FIG. 18;

FIG. 20 is a side elevational, cross-section view of the shaft of the surgical instrument of FIG. 8 showing a routing channel of the shaft for the internal wiring of FIG. 19; and

FIG. 21 is a simplified flow diagram of a method for monitoring operation of the surgical instrument of FIG. 8, which may be executed by the controller of FIG. 8.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, distal, proximal, et cetera, may be used throughout the specification in reference to the surgical instruments described herein as well as in reference to the patient's natural anatomy. Such terms have well-understood meanings in both the study of anatomy and the field of surgery. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (c); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

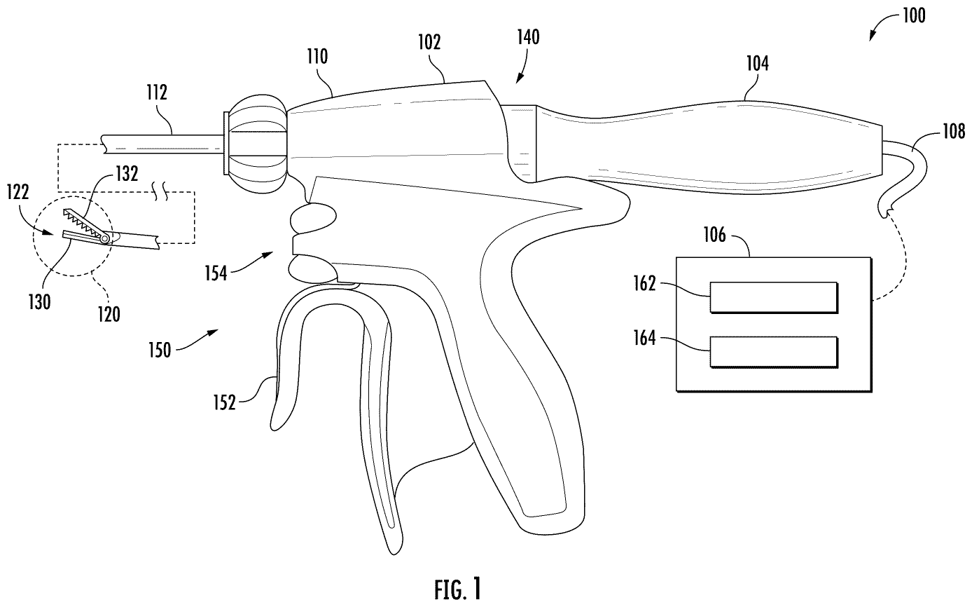

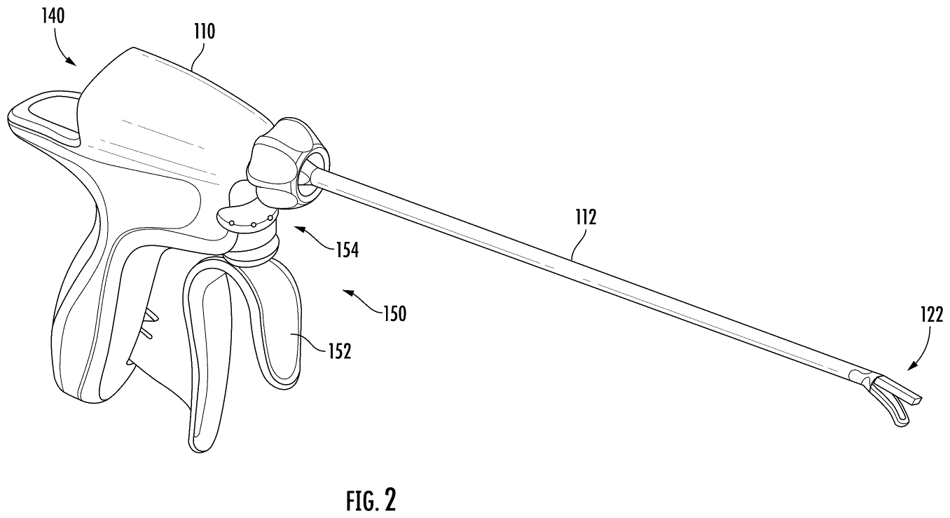

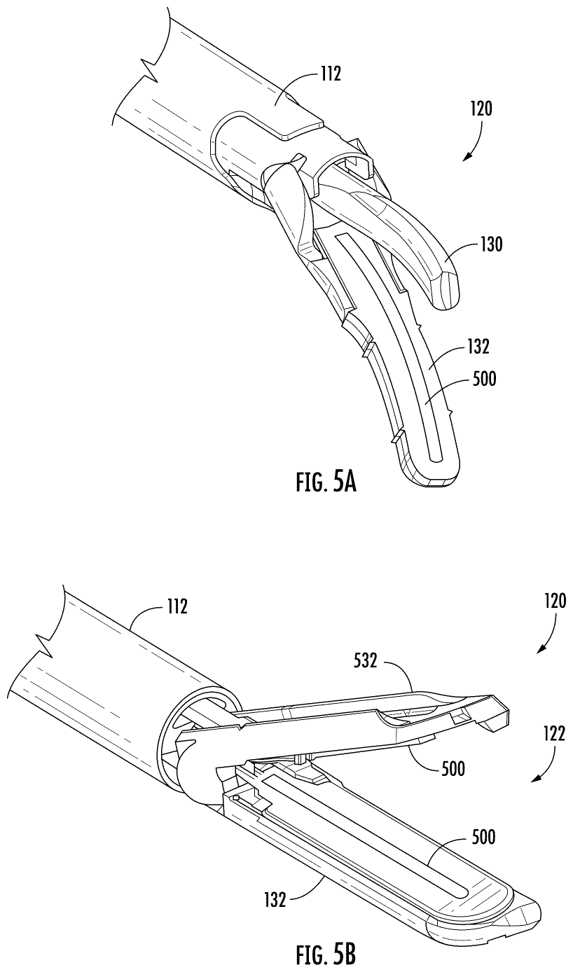

Referring now to FIGS. 1 and 2, in an illustrative embodiment, a system 100 for performing an energy-based surgical procedure includes a surgical instrument 102, a transducer 104, and a generator 106. The surgical instrument 102 is illustratively embodied as an ultrasonic surgical instrument, but may be embodied as an electro-surgical surgical instrument or a multi-modal, ultrasonic/elector-surgical surgical instrument in other embodiments. In use, the surgical instrument 102 is usable to perform various surgical procedures including laparoscopic, endoscopic, or traditional open surgical procedures. In doing so, a surgeon may selectively activate an ultrasonic mode (and/or an electro-surgical/RF mode) of the surgical instrument 102. In the ultrasonic mode, the generator 106 drives the transducer 104 to cause an ultrasonic blade 130 of a jaw assembly 122 of an end effector 120 of the surgical instrument 102 to vibrate at a reference frequency, which facilitates the contemporaneous cutting and hemostatic sealing of patient tissue. Additionally or alternatively, in some embodiments, the surgeon may selectively activate an electro-surgical mode of the surgical instrument 102 to deliver an amount of therapeutic RF energy to the patient tissue to effect hemostatic sealing. In such embodiments, the blade 130 may be embodied as an ultrasonic blade 130 or as a mechanical blade designed to cut tissue using mechanical force (e.g., in those embodiments not employing ultrasonic technologies). Furthermore, in some embodiments, the surgical instrument 102 may be configured with only an electro-surgical/RF mode and, in such embodiments, the jaw assembly 122 of the end effector 120 may not include the ultrasonic blade 130 as discussed in more detail below in regard to FIG. 5B.

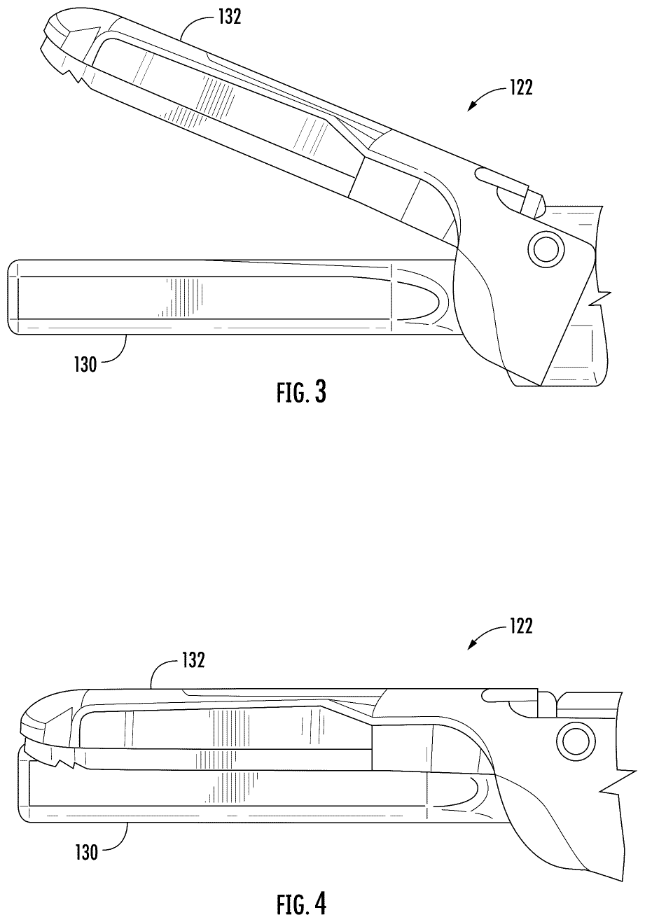

The surgical instrument 102 is illustratively embodied as ultrasonic surgical shears but may be embodied as other types of surgical instruments having an ultrasonic mode and/or electro-surgical mode in other embodiments. In the illustrative embodiment, the surgical instrument 102 includes a handle assembly 110 and an elongated shaft assembly 112, which extends distally away from the handle assembly 110 and may be removably attached to the handle assembly 110 in some embodiments. The elongated shaft assembly 112 includes the end effector 120 located at a distal end opposite the handle assembly 110. The end effector 120 includes the jaw assembly 122, which illustratively includes the ultrasonic blade 130 and a corresponding jaw clamp 132 (but may include two jaw clamps in those embodiments having only an electro-surgical/RF mode). As shown in FIGS. 3 and 4, the jaw assembly 122 is movable between an open state (FIG. 3) in which the jaw clamp 132 is positioned away from the ultrasonic blade 130 and a closed state (FIG. 4) in which the jaw clamp 132 is positioned near or otherwise contacts the ultrasonic blade 130. Actuation of the jaw assembly 122 from the open state to the closed state allows for the grasping, cutting, and coagulation of vessels and/or tissue by the jaw assembly 122. It should be appreciated that the open state may correspond to a degree of openness that is less than a fully opened position of the jaw assembly 122 and the closed state may correspond to a degree of closeness that is less than a fully closed position. That is, the closed state may, for example correspond to a minimal distance between the distal ends of the jaw clamp 132 and the ultrasonic blade 130 and the open state may correspond to a maximum distance between the distal ends of the jaw clamp 132 and the ultrasonic blade 130. However, in other embodiments, the open state may correspond to a fully opened position of the jaw assembly 122 and the closed state may correspond to a fully closed position of the jaw assembly 122.

In those embodiments in which the surgical instrument 102 includes both an ultrasonic mode and an electro-surgical/RF mode, the end effector 120 may include one or more RF electrodes 500 incorporated into the jaw clamp 132 as shown in FIG. 5A. Although the illustrative end effector 120 includes only a single electrode 500 in the embodiment of FIG. 5A, it should be appreciated that the end effector 120 may include additional electrodes 500 in other embodiments (e.g., multiple pads of electrodes 500). The electrode(s) 500 may be embodied as an active electrode configured to the RF energy or as a return electrode configured to “sink” an applied RF energy. In those embodiments utilizing bi-polar RF implementation, the ultrasonic blade 130 may embody the active or return electrode, with the electrode 500 embodying the other active or return electrode. Alternatively, other active or return electrodes may be incorporated on the ultrasonic blade 130 or in another part of the jaw assembly 122 of the end effector 120. In mono-polar implementation, the RF electrode(s) 500 may be embodied as an active electrode, and a return electrode may be attached to a portion of the patient's body.

In those embodiments in which the surgical instrument 102 includes only an electro-surgical/RF mode, the jaw assembly 122 of the end effector 120 includes a jaw clamp 532 in place of the ultrasonic blade 130 as shown in FIG. 5B. In such embodiments, an electrode 500 may be attached to or otherwise incorporated into each jaw clamp 132, 532 and be embodied as an active or a return electrode to facilitate the application of RF energy to tissue captured between the jaw clamps 132, 532. In such embodiments, the surgical instrument 102 may include a knife incorporated into the elongated shaft assembly 112 that is configured to eject outwardly to cut the patient's tissue after sealing of the tissue by the RF energy.

Referring back to FIGS. 1 and 2, in those embodiments including ultrasonic capabilities, the handle assembly 110 includes a receptacle 140 configured to receive the transducer 104 to facilitate connection of the transducer 104 to the handle assembly 110 and the elongated shaft assembly 112. The handle assembly 110 also includes a trigger assembly 150, which includes a primary trigger 152 and a switch assembly 154. The primary trigger 152 is operable by the surgeon to move the jaw assembly 122 of the end effector 120 between the open and closed states. The switch assembly 154 includes one or more buttons, which are selectable by the surgeon to activate (and configure, in some embodiments) the ultrasonic mode and/or the electro-surgical mode of the surgical instrument 102.

The transducer 104 is illustratively connected to the generator 106 by a cable assembly 108. As discussed above, the generator 106 is configured to drive the transducer 104 at a reference or resonant frequency to thereby cause the ultrasonic blade 130 to vibrate. For example, in an illustrative embodiment, the generator 106 may supply an electrical signal to the transducer 104 to cause the ultrasonic blade 130 of the jaw assembly 122 to vibrate longitudinally in the range of, for example, approximately 20 kHz to 250 kHz. In particular embodiments, for example, the ultrasonic blade 130 may vibrate in the range of about 54 kHz to 56 kHz (e.g., at about 55.5 kHz). In other embodiments, the ultrasonic blade 130 may vibrate at other frequencies including, for example, about 31 kHz or about 80 kHz. The excursion of the vibrations at the ultrasonic blade 130 can be controlled by, for example, controlling the amplitude of the electrical signal applied to the transducer 104 by the generator 106. The generator 106 may be activated so that electrical energy may be continuously or intermittently supplied to the transducer 104. The generator 106 also has a power line (not shown) for insertion in an electro-surgical unit or conventional electrical outlet. Additionally or alternatively, the generator 106 may be powered by a direct current (DC) source, such as a battery.

In some embodiments, the generator 106 may be configured to operate in different modes. In such embodiments, the generator 106 may include an ultrasonic generator module 162 for controlling an ultrasonic mode, an electro-surgical/Radio Frequency (RF) generator module 164 for controlling an electro-surgical mode, and/or other generator modules (e.g., a heat generator module) for controlling other operation modes. The various modes of the generator 106 may be operated independently of each other in some embodiments. For example, the generator 106 may activate the ultrasonic mode of the ultrasonic generator module 162 to apply ultrasonic energy to the jaw assembly 122 and subsequently, either therapeutic or sub-therapeutic RF energy may be applied to the jaw assembly 122 by the electro-surgical generator module 164. Alternatively, the activation modes of the generator 106 may be operated simultaneously or contemporaneously with each other.

In the electro-surgical mode, the electro-surgical generator module 164 is configured to generate RF energy at a frequency in the range of about 100 kilohertz (100 kHz) to about 1 megahertz (1 MHz). The generated RF energy is supplied to the patient's tissue via the electrodes 500 of the end effector 120 as described above in regard to FIG. 5. In some embodiments, the electro-surgical generator module 164 may also be configured to selectively provide the RF energy at sub-therapeutic levels to perform various electrical measurements of the patient's tissue. For example, the electro-surgical generator module 164 may be configured to measure an impedance of the patient's tissue using the electrodes 500 and a suitable RF energy level.

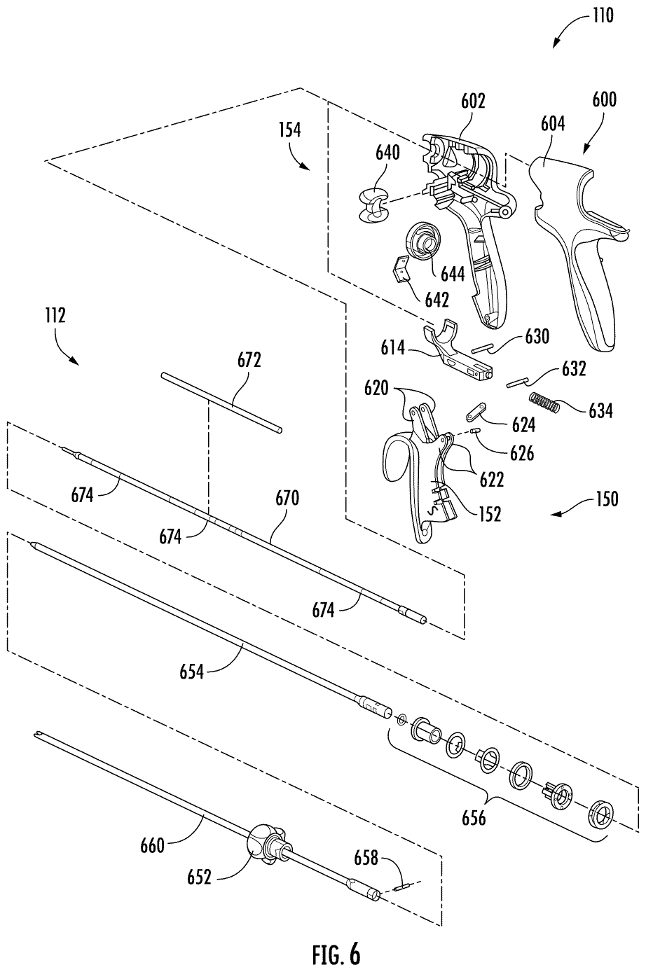

Referring now to FIG. 6, as discussed above, the illustrative surgical instrument 102 includes the handle assembly 110 and the elongated shaft assembly 112, which extends distally away from the handle assembly 110. The handle assembly 110 includes a housing 600, which includes a right half-housing 602 and a left half-housing 604. The half-housings 602, 604 are configured to mate with each other to form the housing 600. To facilitate such mating, each of the half-housings 602, 604 may include various interfaces sized to mechanically align and engage one another to form the housing 600 and enclose the internal working components of the surgical instrument 102.

The primary trigger 152 of the trigger assembly 150 is coupled to a linkage mechanism to translate the rotational motion of the primary trigger 152 to axial motion of a yoke 610, which in turn is configured to move the jaw assembly 122 of the end effector 120 between the open and closed states via the elongated shaft assembly 112. The primary trigger 152 includes a first set of flanges 620 having openings formed therein to receive a first yoke pin 630, which extends through the yoke 610. The primary trigger 152 also includes a second set of flanges 622 configured to receive a first end of a link 624. A trigger pin 626 is received in openings formed in the first end of the link 624 and the second set of flanges 622. The trigger pin 626 forms a trigger pivot point for the primary trigger 152. A second end of the link 624, opposite the first end, is received in a slot formed in a proximal end of the yoke 610 and retained therein by a second yoke pin 632. As the primary trigger 152 is rotated about the pivot point formed from the trigger pin 626, the yoke 610 translates horizontally. A spring 634 is used to bias the yoke forward such that the jaw assembly 122 of the end effector 120 is biased to the open state (or a fully opened state).

As discussed above, the trigger assembly 150 also includes a switch assembly 154. The switch assembly 154 illustratively includes a toggle switch 640, which is selectable to activate one or more switches 642. Activation of the switches 642 electrically energizes an electrical element 644, which electrically energizes the ultrasonic transducer 104 to engage the ultrasonic mode of the surgical instrument 102.

The elongated shaft assembly 112 includes an outer tubular sheath 650 and a rotation knob 652 coupled to the outer cylindrical sheath 650. The rotation knob 652 is operable to rotate the outer cylindrical sheath 650 about an axis defined by the outer cylindrical sheath 650. A reciprocating tubular actuator 654 is located within the outer tubular sheath 650 and mechanically engaged with the end effector 120 on a distal end. The reciprocating tubular actuator 654 is also mechanically engaged, on a proximal end, with the yoke 610 within the handle assembly 110 via coupling elements 656. In embodiments including an ultrasonic mode, an ultrasonic waveguide 670 is located within the reciprocating tubular actuator 654. A distal end of the ultrasonic waveguide 670 is acoustically coupled (e.g., directly or indirectly mechanically coupled) to the ultrasonic blade 130, and a proximal end is acoustically coupled to the transducer 104. The ultrasonic waveguide 670 may be isolated from other components of the elongated shaft assembly 112 by a protective sheath 672 and a number of isolation elements 674. The outer tubular sheath 650, the reciprocating tubular actuator 654, and the ultrasonic waveguide 670 are mechanically engaged together via a pin 658.

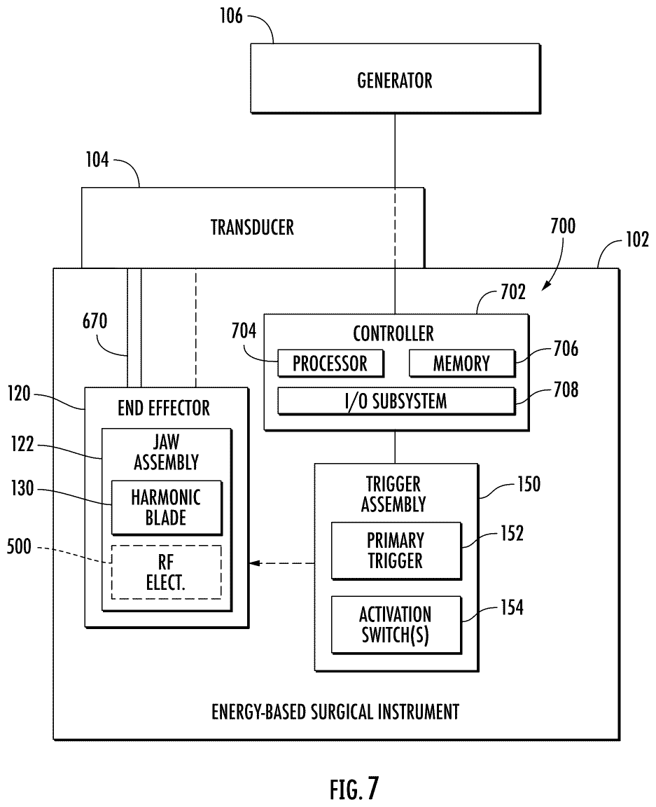

Referring now to FIG. 7, in the illustrative embodiment, the surgical instrument 102 includes a control circuit 700. The control circuit 700 includes a controller 702 and the trigger assembly 150, which cooperate to provide ultrasonic energy to the harmonic blade 130 of the jaw assembly 122 of the end effector 120 and/or RF energy to the RF electrodes 500 of the jaw assembly 122, depending on the operation modes of the surgical instrument 102 as discussed above. In other embodiments, however, the control circuit 700 may include additional or other electronic devices and/or circuit.

The controller 702 may be embodied as any type of controller, functional block, digital logic, or other component, device, circuitry, or collection thereof capable of performing the functions described herein. In illustrative embodiment, the controller 702 includes a processor 704, a memory 706, and an input/output (I/O) subsystem 708. The processor 704 may be embodied as any type of processor capable of performing the functions described herein. For example, the processor 704 may be embodied as a single or multi-core processor(s), digital signal processor, microcontroller, or other processor or processing/controlling circuit. Similarly, the memory 706 may be embodied as any type of volatile and/or non-volatile memory or data storage capable of performing the functions described herein. In operation, the memory 706 may store various data and software used during operation of the control circuit 700 such as executable firmware or software, programs, libraries, and drivers, which may be executed or otherwise used by the processor 704.

The processor 704 and memory 706 are communicatively coupled to other components of the control circuit 700 via the I/O subsystem 708, which may be embodied as circuitry and/or components to facilitate input/output operations between the controller 702 (e.g., the processor 704 and the memory 706) and the other components of the control circuit 700. For example, the I/O subsystem 708 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 708 may form a portion of a system-on-a-chip (SoC) and be incorporated, along with the processor 704 and the memory 706, and other components of the surgical instrument 102, on a single integrated circuit chip. Additionally, in some embodiments, the memory 706, or portions of the memory 706, may be incorporated into the processor 704.

During operation, as discussed above, the controller 702 is configured to control activation of an ultrasonic mode and/or an electro-surgical/RF mode of the surgical instrument 102. To do so, the controller 702 may monitor for activation of the primary trigger 152 and/or one or more activation switches 154 of the trigger assembly 150. In response to activation of the appropriate trigger 152 or switch 154, the controller 702 controls the transducer 104 to generate the ultrasonic energy, which is propagated to the harmonic blade 130 via the ultrasonic waveguide 670. Additionally or alternatively, in response to activation of a corresponding switch 154 of the trigger assembly 150, the controller 702 may be configured to supply an amount of RF energy, via the electro-surgical generator module 164 to the RF electrodes 500 via interconnections 710. It should be appreciated that, although the transducer 104 and the generator 106 are shown as separate components from the energy-based surgical instrument 102 in FIGS. 1 and 7, the transducer 104 and/or the generator 106 may be incorporated into the surgical instrument 102 in other embodiments.

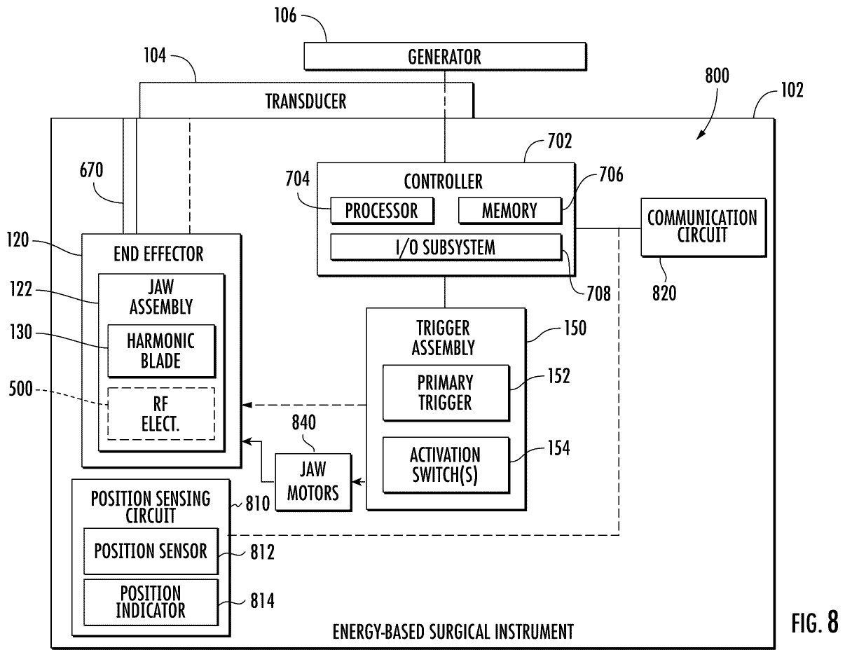

Referring now to FIG. 8, in another embodiment, the surgical instrument 102 includes a position sensing circuit 810 configured to generate sensor data indicative of a present position of the jaw assembly 122 (e.g., a position between the open state and the closed state of the jaw assembly 122). To do so, the position sensing circuit 810 illustratively includes a position sensor 812 and an associated position indicator 814. In use, as discussed in more detail below, the position sensor 812 is configured to sense movement of the position indicator 814 during operation of the trigger assembly 150 to move the jaw assembly 122. In response to the sensed movement of the position indicator, the position sensor 812 is configured to generate sensor data indicative of the present position of the jaw assembly 122. The controller 702 may control operation of the surgical instrument 102 based on the determined present position of the jaw assembly 122 and/or transmit the sensor data to the generate 106 for further processing or use.

In such embodiments, the surgical instrument 102 may include a control circuit 800 as shown in FIG. 8. The control circuit 800 is substantially similar to the control circuit 700 described above and components of the control circuit 800 that correspond to similar components of control circuit 700 have been identified using the same reference number in FIG. 8 with the understanding that the description of such components provided above with regard to FIG. 7 applies to the corresponding components of the control circuit 800.

As shown in FIG. 8, the control circuit 800 includes the position sensing circuit 810 and a communication circuit 820. As described above, the illustrative position sensing circuit 810 includes one or more position sensors 812 and one or more position indicators 814. The position sensor 812 may be embodied as any type of sensor or sensor device capable of sensing movement and/or relative position of the position indicator 814. For example, as described in more detail below, the position sensor 812 may be embodied as a camera, an optocoupler, a magnetic sensor, and/or other sensor or sensor circuit or device. Similarly, the position indicator 814 may be embodied as markings or other indicia visible by a camera, a reflective surface detectable by an optocoupler, a magnet detectable by a magnetic sensor, and/or other feature the movement and/or position of which is detectable by a corresponding position sensor 812.

As described in more detail below, in use, the position indicator 814 is configured to move during operation of the trigger assembly 150 to move the jaw assembly 122 to a desired state (e.g., between an open state and a closed state). Movement of the position indicator is detected by the corresponding position sensor 812, which produces sensor data that is indicative of the present position of the jaw assembly 122. That is, the movement of the jaw assembly 122 causes movement of the position indicator 814, which is detected by the position sensor 812. In this way, the sensor data produced by the position sensor 312 corresponds to or otherwise indicative of the present position of the jaw assembly 122.

The communication circuit 820 of the control circuit 800 may be embodied as any type of communication circuit, device, or collection thereof, capable of enabling communications between the surgical instrument 102 and the remote generator 106 and/or other remote devices. To do so, the communication circuit 820 may utilize any suitable communication protocol including, but not limited to, Ethernet, Wi-Fi (e.g., communications based on the Institute of Electrical and Electronics Engineers (IEEE) 802.11 family), a proprietary protocol, and/or other communication protocols.

Referring now to FIGS. 9-11, in some embodiments, the position sensor 812 may be embodied as a camera 912 mounted internally in the surgical instrument 102 and configured to detect movement of the position indicator 814. The position indicator 814 is attached to or defined on an internal component of the surgical instrument 102 that moves in response to activation of the trigger assembly 150 to move the jaw assembly 122. For example, in the illustrative embodiment of FIG. 9, the position indicator 814 is embodied as indica 914 defined on an internal shaft 950 of the surgical instrument 102. The indicia 914 is located within a view 960 of the camera 912. However, in response to operation of the trigger assembly 150 to move the jaw assembly 122, an internal sleeve 952 of the trigger assembly 150 is moved to obscure or cover a portion of the indicia 914. The covering of the indica 914 is detectable by the camera 912, and the amount of coverage of the indicia 914 corresponds to the position of the jaw assembly 122.

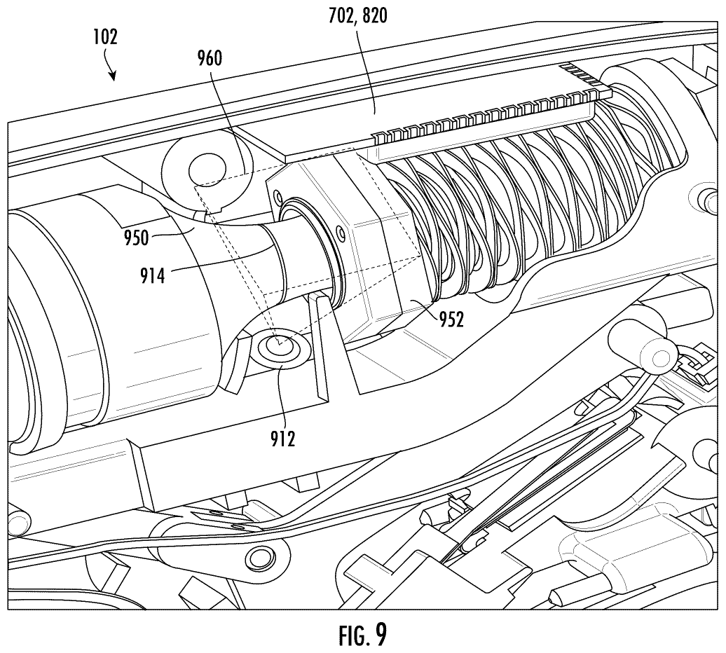

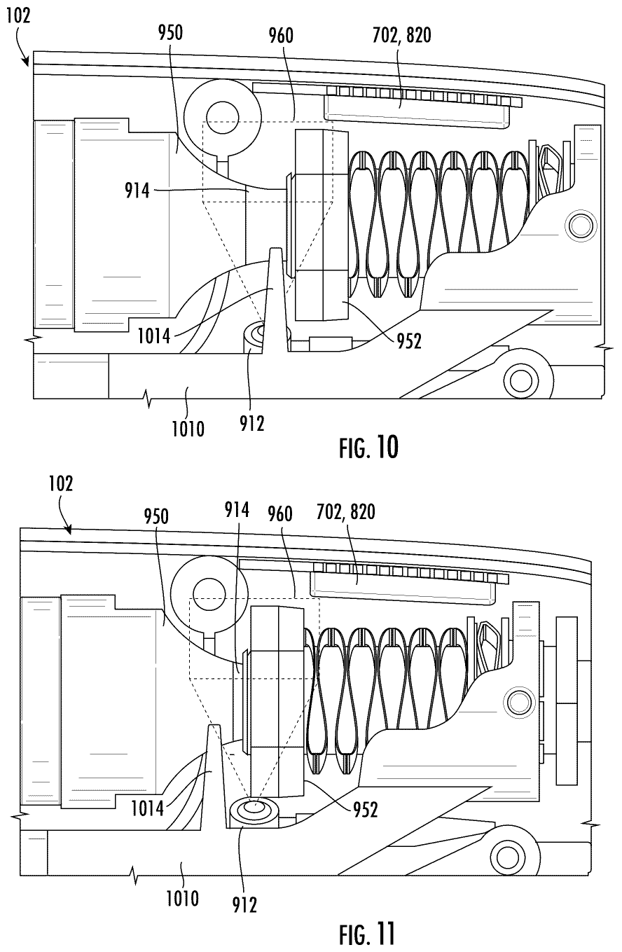

For example, as shown in FIG. 10, when the trigger assembly 150 is not engaged and the jaw assembly 122 is in the fully open state, the entirety of the indicia 914 (or a large portion thereof) is visible to the camera. However, as shown in FIG. 11, when the trigger assembly 150 is operated to move the jaw assembly 122, the internal sleeve 952 is moved proximally and covers a portion of the indicia 914. As discussed above, the amount of the indicia 914 covered by the internal sleeve 952 is indicative of the present position of the jaw assembly 122 and is detected by the camera 912.

In some embodiments, the position indicator 814 may also include a yoke arm 1014 of a yoke 1010 of the surgical instrument 102. In such embodiments, the yoke arm 1014 is also positioned within the view 960 of the camera 912. Similar to the internal sleeve 952, the yoke 1010 moves proximally in response to activation of the trigger assembly 150 to move the jaw assembly 122. As the yoke 1010 moves, the yoke arm 1014 also moves within the view 960 of the camera 912 as shown comparatively in FIGS. 10 and 11. The movement and/or position of the yoke arm 1014 within the view 960 of the camera 912 is indicative of the present amount of clamp force of the jaw assembly 122. As such, the camera 912 is configured to generate sensor data that is indicative of the clamp force of the jaw assembly 122 based on the sensed movement and/or position of the yoke arm 1014.

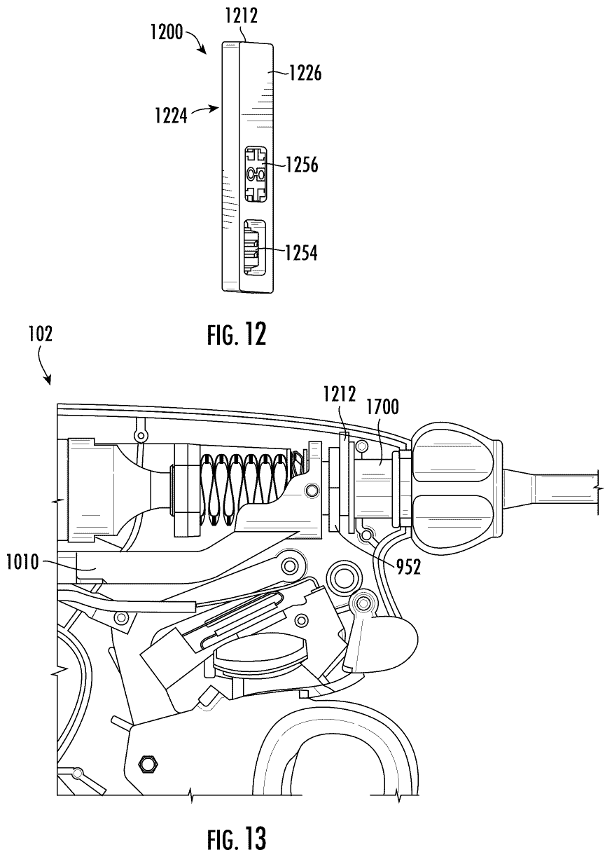

Referring now to FIGS. 12-17, in other embodiments, the position sensor 812 may be embodied as a optocoupler sensor array 1200. The illustrative optocoupler sensor array 1200 includes a sensor body 1212 having a proximal side 1224 and a distal side 1226 opposite the proximal side 1224. The optocoupler sensor array 1200 also includes an optocoupler sensor 1254 located on the proximal side 1224 of the sensor body 1212 and an optocoupler sensor 1256 located on the distal side 1226 of the sensor body 1212.

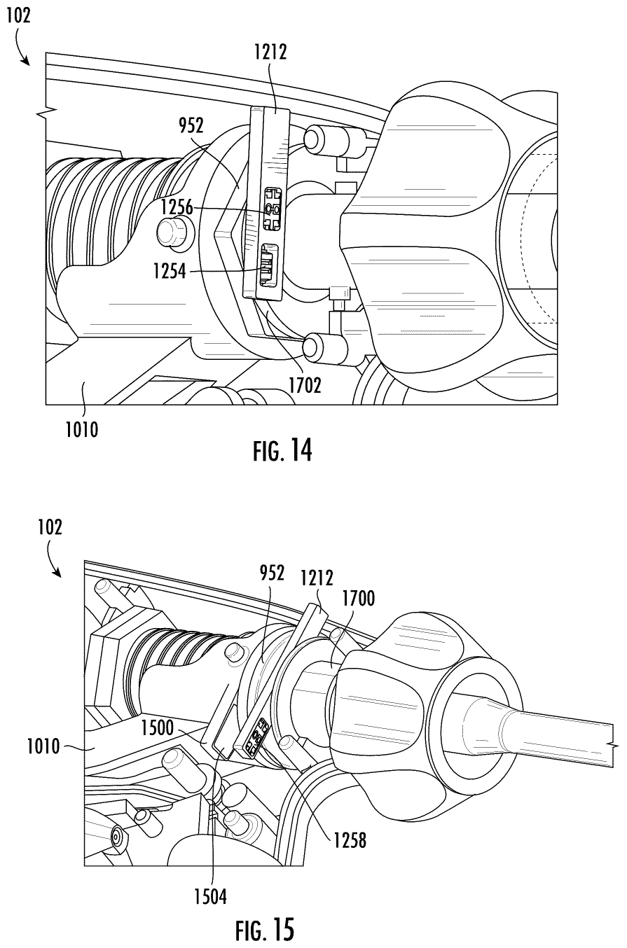

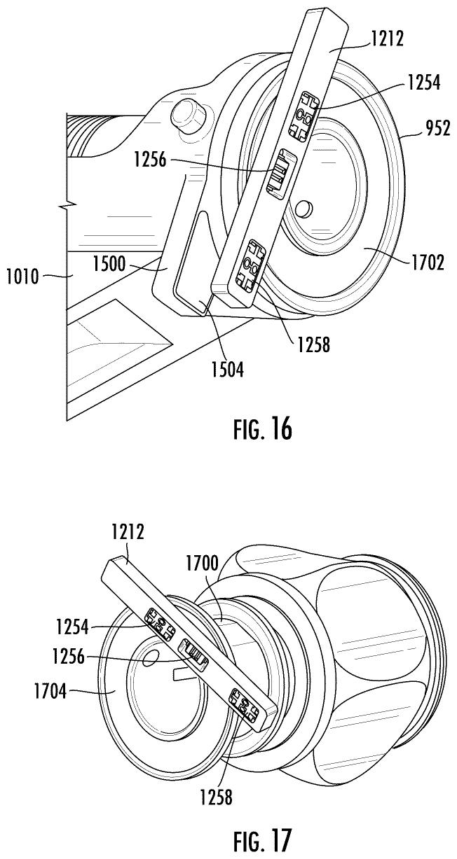

As shown in FIGS. 13 and 14, the optocoupler sensor array 1212 is configured to be mounted internally within the surgical instrument 102 in a position at which each optocoupler sensor 1254, 1256 is capable of monitoring the movement and/or location of a corresponding position indicator 814. For example, as shown in FIGS. 14 and 16, the optocoupler 1254 is configured to sense movement of a position indicator 814 embodied as a reflective ring 1404 attached to the inner sleeve 952 of the surgical instrument 102. Similarly, as best shown in FIG. 17, the optocoupler 1256 is configured to sense movement of a position indicator 814 embodied as a reflective ring 1704 attached to an inner shaft 1700 of the surgical instrument.

Each of the reflective rings 1702, 1704 is configured to move, relative to their corresponding optocoupler 1254, 1256, in response to operation of the trigger assembly 150 to move the jaw assembly 122. That is, in response to operation of the trigger assembly 150, the internal sleeve 952 is moved proximally, which causes movement of the reflective ring 1702. The movement of the reflective ring 1702 is detected by the optocoupler 1254, which generates sensor data indicative of the present position of the jaw assembly 122 based on the sensed movement and/or position of the reflective ring 1702. Similarly, in response to operation of the trigger assembly 150, the internal shaft 1700 is moved proximally, which causes movement of the reflective ring 1704. The movement of the reflective ring 1704 is detected by the optocoupler 1256, which generates sensor data indicative of the present position of the jaw assembly 122 based on the sensed movement and/or position of the reflective ring 1704.

In some embodiments, the optocoupler sensor array 1212 may also include an optocoupler sensor 1258 located on the proximal side 1224 of the sensor body 1212 as shown in FIGS. 15-16. Similarly to the optocouplers 1254, 1256, the optocoupler sensor 1258 is capable of monitoring the movement and/or location of a corresponding position indicator 814. For example, as shown in FIGS. 15 and 16, the optocoupler 1258 is configured to sense movement of a reflective surface 1504 defined on a yoke arm 1500 of the yoke 1010 of the surgical instrument 102. That is, when the trigger assembly 150 is operated to move the jaw assembly 122, the yoke 1010 is moved proximally, which causes movement of the yoke arm 1500 and the associated reflective surface 1504, relative to the optocoupler 1258. Movement and/or position of the yoke arm 1500, relative to the optocoupler 1258, is indicative of the present amount of clamp force of the jaw assembly 122. As such, the optocoupler 1258 is configured to generate sensor data that is indicative of the clamp force of the jaw assembly 122 based on the sensed movement and/or position of the yoke arm 1500.

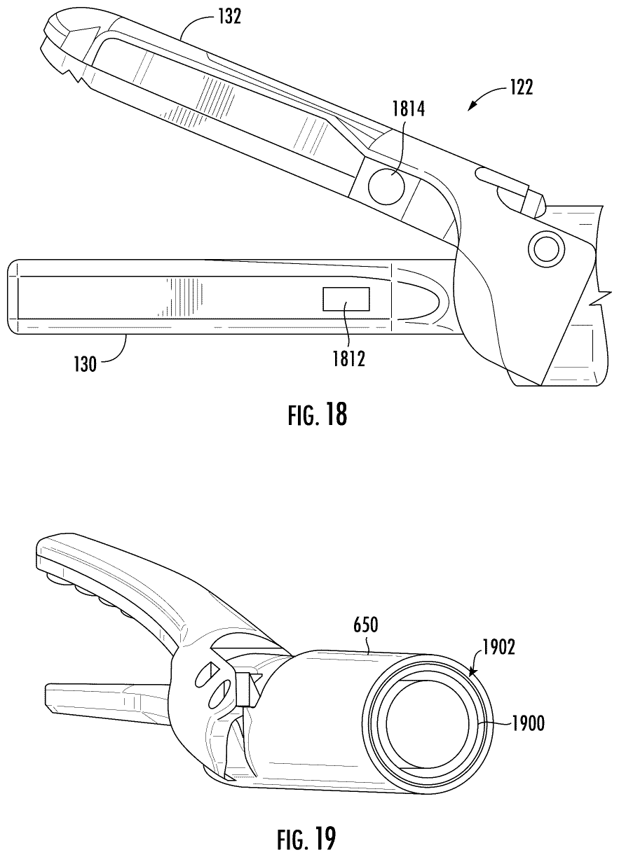

Referring now to FIGS. 18-20, in yet other embodiments, the position sensor 812 may be embodied as a magnetic sensor 1812 and the position indicator may be embodied as a magnetic 1814. Illustratively, the magnetic sensor 1812 is attached to or otherwise embedded in the ultrasonic blade 130 of the jaw assembly 122 and the magnetic is attached to the jaw clamp 132 of the jaw assembly 122. However, it should be appreciated that, in other embodiments, the magnetic sensor 1812 may be attached to the jaw clamp 132 and the magnetic 1814 may be attached to or otherwise embedded in the ultrasonic blade 130. In use, the magnetic sensor 1812 is configured to sense the movement and/or position of the magnet 1814 while the jaw assembly 122 is moved between the open state and the closed state. The sensed distance of the magnet 1814 from the magnetic sensor 182 is indicative of the present position of the jaw assembly 122. As such, the magnetic sensor 1812 is configured to produce sensor data indicative of the position of the jaw assembly 122 based on the sensed position/movement of the magnet 1814.

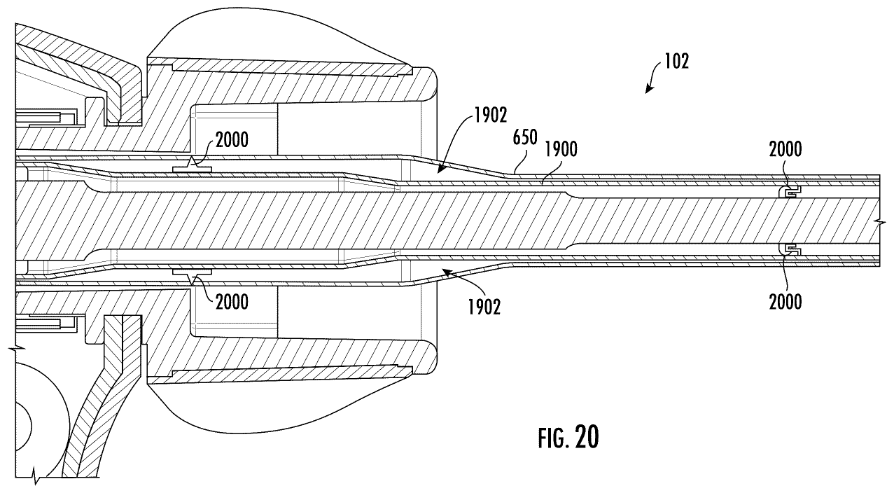

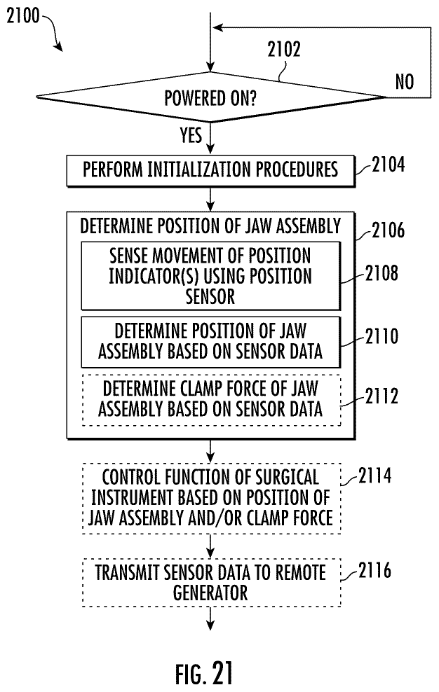

It should be appreciated that routing electrical connections, such as wiring, to the magnetic sensor 1812 from the internal electrical components located in the housing 600 (see FIG. 6) can be challenging due to movement of an internal shaft 1900 (e.g., the reciprocating tubular actuator 654) within the outer sheath 650. As such, as shown in FIGS. 19 and 20, the electrical interconnects connecting the magnetic sensor 1812 are routed within a void 1902 defined between the internal shaft 1900 and the outer sheath 650. To ensure local biomatter is not received within the void 1902, the surgical instrument may include a set of collar or retainer-type seals 2000 within the void 1902 that are configured to allow passage of the electrical connection while limiting or preventing passage of biomatter.

Referring now to FIG. 21, in use, the controller 702 (see FIG. 8) of the surgical instrument 102 may execute method 2100 for monitoring operation of the surgical instrument 102. The method 2100 begins with block 2102 in which the controller 702 determines whether the surgical instrument 102 has been powered to an “on” state. If so, the method 2100 advances to block 2104 in which the controller 702 performs one or more initialization procedures. For example, the controller 702 may verify operation of the position sensing circuit, connection of the transducer 104 and/or remote generator 106, and/or other initialization or verification procedure.

After the controller 702 has performed the initialization procedures in block 2104, the method 2100 advances to block 2106 in which the controller 702 determines a present position of the jaw assembly 122 of the surgical instrument 102. To do so, in block 2108, the position sensor(s) 812 senses movement and/or the relative position of the position indicator(s) 814. As discussed above, the movement and/or position of the position indicator(s) 814 is indicative of the present position of the jaw assembly 122 (e.g., between the closed state and the open state). As such, each position sensor 812 is configured to generate or produce sensor data indicative of the present position of the jaw assembly 122 based on the sensed movement/position of the corresponding position indicator 814.

In block 2110, the controller 702 determines the present position of the jaw assembly 122 based on the sensor data produced by the position sensor(s) 812. Additionally, in some embodiments in block 2112, the controller 702 may be configured to determine an amount of clamp force of the jaw assembly 122 based on the sensor data received from the position sensor(s) 814 as discussed above.

In some embodiments, in block 2114, the controller 702 may be configured to control one or more functions of the surgical instrument 102 based on the position of the jaw assembly 102 determined in block 2106. Additionally or alternatively, in some embodiments in block 2116, the controller 702 may be configured to transmit the sensor data to the remote generator 106 via the communication circuit 820.

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. A surgical instrument for performing an energy-based surgical procedure, the surgical instrument comprising:
an end effector having a jaw assembly movable between an open state and a closed state;
a trigger assembly operable to move the jaw assembly of the end effector between the open state and the closed state; and
a position sensing circuit including a position sensor and a position indicator, wherein the position sensor is configured to sense movement of the position indicator and generate sensor data indicative of a position of the jaw assembly based on the sensed movement of the position indicator.

2. The surgical instrument of claim 1, further comprising a controller configured to determine the position of the jaw assembly based on the sensor data and control a function of the surgical instrument based on the determined position of the jaw assembly.

3. The surgical instrument of claim 1, further comprising a communication circuit configured to transmit the sensor data to a generator remote from the surgical instrument.

4. The surgical instrument of claim 1, wherein the position sensor of the position sensing circuit comprises a camera configured to monitor movement of the position indicator.

5. The surgical instrument of claim 4, wherein the position indicator comprises indica located in view of the camera.

6. The surgical instrument of claim 5, wherein the indicia comprises a marking located on an inner shaft of the surgical instrument, wherein at least a portion of the marking is covered from the view of the camera when the trigger assembly is operated to move the jaw assembly of the end effector.

7. The surgical instrument of claim 4, wherein the trigger assembly further comprises a yoke configured to move in response to operation of the trigger assembly to move the jaw assembly of the end effector,
wherein the position indicator comprises a yoke arm extending upwardly from a yoke body of the yoke and wherein the yoke arm is in view of the camera and moves within the view of the camera in response to operation of the trigger assembly,
wherein the position of the yoke arm within the view of the camera is indicative of an amount of clamp force of the jaw assembly of the end effector.

8. The surgical instrument of claim 4, further comprising an inner shaft and wherein the wherein the trigger assembly further comprises a yoke configured to move in response to operation of the trigger assembly to move the jaw assembly of the end effector,
wherein the position indicator comprises a first position indicator and the first position indicator comprises a marking located on the inner shaft, wherein at least a portion of the marking is covered from the view of the camera when the trigger assembly is operated to move the jaw assembly of the end effector, and
further comprising a second position indicator, wherein the second position indicator comprise a yoke arm extending upwardly from a yoke body of the yoke and wherein the yoke arm is in view of the camera and moves within the view of the camera in response to operation of the trigger assembly,
wherein the position of the yoke arm within the view of the camera is indicative of an amount of clamp force of the jaw assembly of the end effector.

9. The surgical instrument of claim 1, wherein the position sensor of the position sensing circuit comprises an optocoupler configured to monitor movement of a reflective surface.

10. The surgical instrument of claim 1, wherein the position sensor comprises a sensor body, a first optocoupler mounted on a first side of the sensor body and a second optocoupler mounted on a second side of the sensor body.

11. The surgical instrument of claim 10, wherein the position indicator comprises a first reflective ring mounted to an internal sleeve and in view of the first optocoupler and a second reflective ring mounted to an internal shaft and in view of the second optocoupler,
wherein the first reflective ring is configured to move relative to the first optocoupler when the trigger assembly is operated to move the jaw assembly of the end effector and wherein the first optocoupler is configured to sense a distance to the first reflective ring,
wherein the second reflective ring is configured to move relative to the second optocoupler when the trigger assembly is operated to move the jaw assembly of the end effector and wherein the second optocoupler is configured to sense a distance to the second reflective ring.

12. The surgical instrument of claim 11, wherein the trigger assembly further comprises a yoke configured to move in response to operation of the trigger assembly to move the jaw assembly of the end effector,
wherein the position sensor further comprises a third optocoupler mounted to the first side of the sensor body and the position indicator comprises a reflective surface of the yoke,
wherein the third optocoupler is configured to sense a distance to the reflective surface of the yoke.

13. The surgical instrument of claim 1, wherein the position sensor of the position sensing circuit comprises a magnetic sensor and the position indicator comprises a magnet.

14. The surgical instrument of claim 13, wherein the jaw assembly comprises a jaw clamp and a harmonic blade, wherein one of the magnetic sensor and the magnet is attached to the jaw clamp and the other one of the magnetic sensor and the magnet is attached to the harmonic blade.

15. A method comprising:
sensing, by a position sensor, movement of a position indicator during operation of a trigger assembly of a surgical instrument to move the jaw assembly between an open state and a closed state, wherein the position sensor and the position indicator are attached to the surgical instrument;
generating, by the position sensor, sensor data indicative of a position of the jaw assembly based on the sensed movement of the position indicator; and
determining, by a controller of the surgical instrument, the position of the jaw assembly based on the sensor data.

16. The method of claim 15, wherein the position sensor of the position sensing circuit comprises a camera configured to monitor movement of the position indicator and the position indicator comprises indica located in view of the camera.

17. The method of claim 16, wherein the indicia comprises a marking located on an inner shaft of the surgical instrument, wherein at least a portion of the marking is covered from the view of the camera when the trigger assembly is operated to move the jaw assembly of the end effector.

18. The method of claim 15, wherein the position sensor of the position sensing circuit comprises an optocoupler configured to monitor movement of a reflective surface.

19. The method of claim 15, wherein the position sensor comprises a sensor body, a first optocoupler mounted on a first side of the sensor body and a second optocoupler mounted on a second side of the sensor body,
wherein the position indicator comprises a first reflective ring mounted to an internal sleeve and in view of the first optocoupler and a second reflective ring mounted to an internal shaft and in view of the second optocoupler,
wherein the first reflective ring is configured to move relative to the first optocoupler when the trigger assembly is operated to move the jaw assembly of the end effector and wherein the first optocoupler is configured to sense a distance to the first reflective ring, and
wherein the second reflective ring is configured to move relative to the second optocoupler when the trigger assembly is operated to move the jaw assembly of the end effector and wherein the second optocoupler is configured to sense a distance to the second reflective ring.

20. The method of claim 15, wherein the jaw assembly comprises a jaw clamp and a harmonic blade, wherein the position sensor of the position sensing circuit comprises a magnetic sensor attached to one of the jaw clamp and the harmonic blade, and wherein the position indicator comprises a magnet attached to the other one of the jaw clamp and the harmonic blade.
